(19) United States
(12) Patent Application Publication
(10) Pub. No.: US 2026/0283679 A1
(43) Pub. Date: Sep. 24, 2026

(54) SURGICAL END EFFECTOR ASSEMBLIES AND SURGICAL INSTRUMENTS FOR ENERGY-BASED TISSUE CUTTING

(71) Applicant: Covidien LP, Mansfield, MA (US)

(72) Inventors: Dylan R. Kingsley, Broomfield, CO (US); Daniel W. Mercier, Erie, CO (US); Jake H. Young, Erie, CO (US); Craig V. Krastins, Arvada, CO (US); Chelsea E. Walbridge, Longmont, CO (US); David J. Van Tol, Boulder, CO (US)

(21) Appl. No.: 19/477,538

(22) PCT Filed: Apr. 10, 2024
(86) PCT No.: PCT/IB2024/053512

§ 371 Date: Oct. 22, 2025

Related U.S. Application Data
(60) Provisional application No. 63/462,693, filed on Apr. 28, 2023.

(51) Int. Cl. A61B 18/14; A61B 18/00; A61B 34/30

(52) U.S. Cl. CPC A61B 18/1442; A61B 34/30; A61B 2018/00601; A61B 2018/1452

(57) ABSTRACT

A surgical end effector assembly includes first and second jaw members configured to grasp tissue therebetween. The first jaw member includes a compression pad and the second jaw member includes a cutting electrode to enable grasping tissue between the cutting electrode and the compression pad. The compression pad is configured to facilitate cutting tissue grasped between the cutting electrode and the compression pad.

### CROSS REFERENCE TO RELATED APPLICATION

This application claims the benefit of U.S. Provisional Patent Application Ser. No. 63/462,693, filed Apr. 28, 2023, the entire content of which is incorporated herein by reference.

### FIELD

This disclosure relates to surgical instruments and, more specifically, to surgical end effector assemblies and surgical instruments for energy-based tissue cutting such as, for example, for use in surgical robotic systems.

### BACKGROUND

Surgical robotic systems are increasingly utilized in various different surgical procedures. Some surgical robotic systems include a console supporting a robotic arm. One or more different surgical instruments may be configured for use with the surgical robotic system and selectively mountable to the robotic arm. The robotic arm provides one or more inputs to the mounted surgical instrument to enable operation of the mounted surgical instrument.

A surgical forceps, one type of instrument capable of being utilized with a robotic surgical system, relies on mechanical action between its jaw members to grasp, clamp, and constrict tissue. Electrosurgical forceps utilize both controlled mechanical clamping action and energy to heat tissue to seal (or otherwise treat) tissue. Typically, once tissue is sealed, the tissue is severed using a cutting element. Accordingly, many electrosurgical forceps are designed to incorporate a mechanical cutting element to effectively sever sealed tissue (and/or to cut tissue independently of tissue sealing). Alternatively, surgical forceps may incorporate an energy-based, e.g., thermal, electrical, ultrasonic, etc., cutting mechanism to cut tissue, whether previously sealed or unsealed.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of this disclosure is a surgical end effector assembly including first and second jaw members having respective first and second tissue contacting surfaces. At least one of the first or second jaw members is movable relative to the other of the first or second jaw members between a spaced apart position and an approximated position for grasping tissue between the first and second tissue contacting surfaces. The second jaw member includes a cutting electrode extending from the second jaw member towards the first jaw member. The first jaw member includes a compression pad configured to oppose the cutting electrode in the approximated position of the first and second jaw members for grasping tissue between the cutting electrode and the compression pad in the approximated position of the first and second jaw members. The compression pad includes a first portion having a first durometer and a second portion having a second durometer different from the first durometer.

In an aspect of this disclosure, the first and second portions of the compression pad are vertically stacked such that the first portion is configured to contact tissue while the second portion is substantially unexposed within the first jaw member.

In another aspect of this disclosure, the first and second portions of the compression pad are longitudinally aligned such that the first portion defines a proximal compression pad portion and the second portion defines a distal compression pad portion.

In still another aspect of this disclosure, the first and second portions of the compression pad are laterally aligned such that the first portion defines a right compression pad portion and the second portion defines a left compression pad portion.

In yet another aspect of this disclosure, the first portion of the compression pad includes first and second outer sections and the second portion of the compression pad is disposed between the first and second outer sections.

In still yet another aspect of this disclosure, the first portion includes a body of the compression pad and the second portion includes a plurality of voids defined through the body of the compression pad. In such aspects, at least one void of the plurality of voids may be filled with a material different from a material forming the body of the compression pad.

In an aspect of this disclosure, the first portion is a first overmold and the second portion is a second overmold.

In another aspect of this disclosure, the second portion surrounds at least a portion of the first portion.

In yet another aspect of this disclosure, the first portion includes a first filler material and the second portion includes a second, different filler material or no filler material.

Another surgical end effector assembly provided in accordance with this disclosure includes first and second jaw members having respective first and second tissue contacting surfaces. At least one of the first or second jaw members is movable relative to the other of the first or second jaw members between a spaced apart position and an approximated position for grasping tissue between the first and second tissue contacting surfaces. The second jaw member includes a cutting electrode extending from the second jaw member towards the first jaw member. The first jaw member includes a compression pad disposed within a slot defined through the first tissue contacting surface. The compression pad is configured to oppose the cutting electrode in the approximated position of the first and second jaw members for grasping tissue between the cutting electrode and the compression pad with the cutting electrode at least partially compressing the compression pad from an initial condition to a compressed condition in the approximated position of the first and second jaw members. In the initial condition, a portion of the compression pad is spaced apart from a wall defining the slot. In the compressed condition, the portion of the compression pad is urged towards and into contact with the wall defining the slot.

In an aspect of this disclosure, the slot includes at least one relief recess. In such aspects, the wall defining the slot may be disposed within the relief recess. Further, in aspects, the at least one recess may include a pair of relief recesses disposed on either side of the compression pad.

In another aspect of this disclosure, the wall is a lateral wall of the slot.

In still another aspect of this disclosure, the compression pad defines a volume less than a volume of the slot such that, in the initial condition, a pocket is defined between the compression pad and the wall defining the slot.

A surgical instrument provided in accordance with this disclosure includes a housing, a shaft assembly extending distally from the housing, and an end effector assembly extending distally from the shaft assembly. The end effector assembly includes first and second jaw members including respective first and second tissue contacting surfaces. At least one of the first or second jaw members is movable relative to the other of the first or second jaw members between a spaced apart position and an approximated position for grasping tissue between the first and second tissue contacting surfaces. The second jaw member includes a cutting electrode extending from the second jaw member towards the first jaw member. The first jaw member includes a compression pad configured to oppose the cutting electrode in the approximated position of the first and second jaw members for grasping tissue disposed between the cutting electrode and the compression pad in the approximated position of the first and second jaw members. The compression pad defines a varied compressibility in at least one dimension of the compression pad.

In an aspect of this disclosure, the compression pad includes portions formed from different materials, thereby defining the varied compressibility.

In another aspect of this disclosure, the compression pad is at least partially disposed within a slot defined within the first jaw member. The slot defines a varied width to thereby vary a width of the compression pad and define the varied compressibility.

In still another aspect of this disclosure, the compression pad is at least partially disposed within a slot defined within the first jaw member. The varied compressibility is defined by a first portion of the compression pad extending into a previously unoccupied portion of the slot in response to compression of the compression pad and a second portion of the compression pad being urged against a wall of the slot in response to the compression of the compression pad.

In yet another aspect of this disclosure, the varied compressibility at least partially corresponds to a variation in force applied to tissue grasped between the cutting electrode and the compression pad in the approximated position of the first and second jaw members.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a surgical instrument provided in accordance with the present disclosure configured for mounting on a robotic arm of a surgical robotic system such as the surgical robotic system of FIG. 1;

FIGS. 6A and 6B are front and rear perspective views, respectively, of a proximal portion of the surgical instrument of FIG. 5, with an outer shell removed;

FIG. 7 is a front perspective view of the proximal portion of the surgical instrument of FIG. 5 with the outer shell and additional internal components removed;

FIGS. 8A and 8B are side views of a portion of the end effector assembly of the surgical instrument of FIG. 5 with jaw members of the end effector assembly disposed in spaced apart and approximated positions, respectively;

FIG. 9 is a transverse, cross-sectional view of the end effector assembly of the surgical instrument of FIG. 5;

FIG. 10A is a plan view of one of the jaw members of the end effector assembly of the surgical instrument of FIG. 5;

FIG. 10B is a plan view of the other jaw member of the end effector assembly of the surgical instrument of FIG. 5;

FIGS. 11-19 are perspective views of various different configurations of compression pads according to aspects of this disclosure and configured for use with the end effector assembly of the surgical instrument of FIG. 5;

FIGS. 20-29 are transverse, cross-sectional views of one of the jaw members of the end effector assembly of the surgical instrument of FIG. 5 including various different configurations of compression pads disposed therein according to aspects of this disclosure; and

FIG. 30 is a side view of another compression pad in accordance with aspects of this disclosure in use with the end effector assembly of the surgical instrument of FIG. 5, wherein the jaw members of the end effector assembly are disposed in a spaced apart position.

### DETAILED DESCRIPTION

This disclosure provides surgical end effector assemblies and surgical instruments for energy-based tissue cutting. As described in detail below, the surgical end effector assemblies and surgical instruments of this disclosure are configured for use with a surgical robotic system, which may include, for example, a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which, in turn, move the robotic arm in response to the movement command. Those skilled in the art will understand that this disclosure, although described in connection with surgical robotic systems, may also be adapted for use with handheld surgical instruments such as, for example, endoscopic surgical instruments and/or open surgical instruments, whether manually operated or powered.

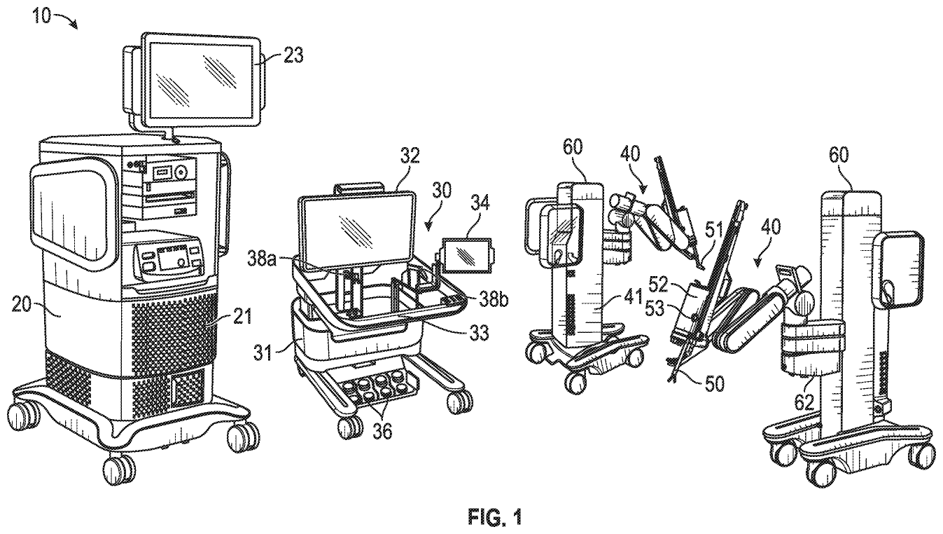

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50, 51 removably coupled thereto. Each of the robotic arms 40 is also coupled to a movable cart 60.

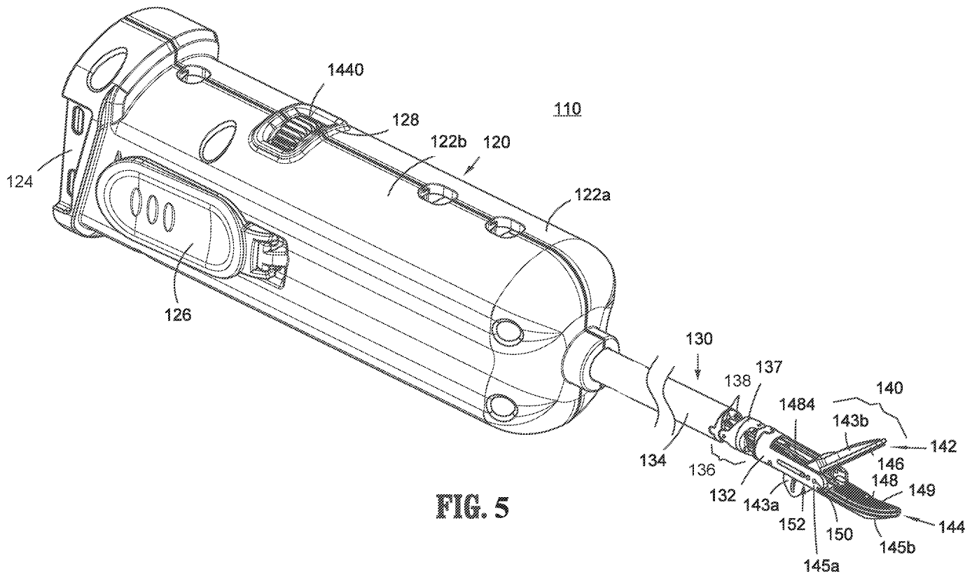

The one or more surgical instruments 50, 51 may be configured for use during minimally invasive surgical procedures and/or open surgical procedures. In aspects, one of the surgical instruments 50 may be an endoscope, such as an endoscope camera 51, configured to provide a video feed for the clinician. In aspects, one of the surgical instruments 50 may be an energy based surgical instrument such as, for example, an energy-based forceps configured to seal tissue by grasping tissue between opposing structures and applying energy, e.g., electrical, thermal, ultrasonic, light, etc., energy thereto and to cut tissue by applying energy, e.g., electrical, thermal, ultrasonic, light, etc., energy thereto. An example of such an energy-based forceps for energy-based sealing and cutting is described in detail below and identified by reference numeral 110 (FIG. 5).

Endoscope camera 51 is configured to capture video of the surgical site. The surgical console 30 includes a first display 32, which displays a video feed of the surgical site provided by endoscope camera 51, and a second display 34, which displays a user interface for controlling the surgical robotic system 10. The first and second displays 32 and 34 are touchscreens allowing for display of and interaction with various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, 51 based on a set of programmable instructions and/or input commands from the surgical console 30, in such a way that robotic arms 40 and the surgical instruments 50, 51 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

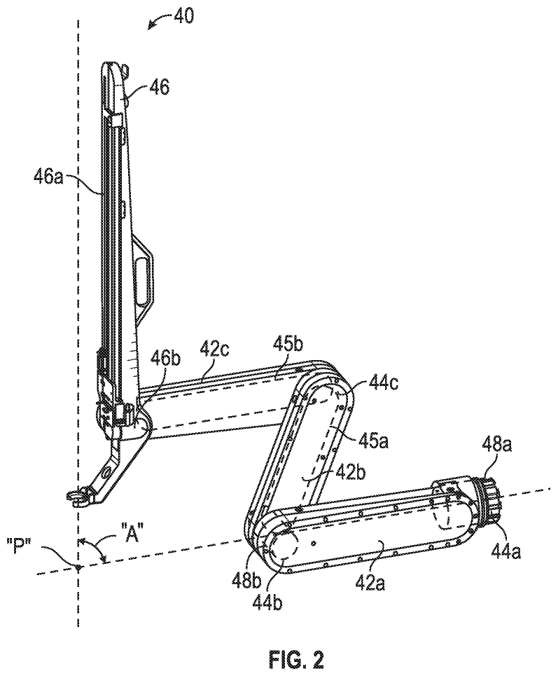

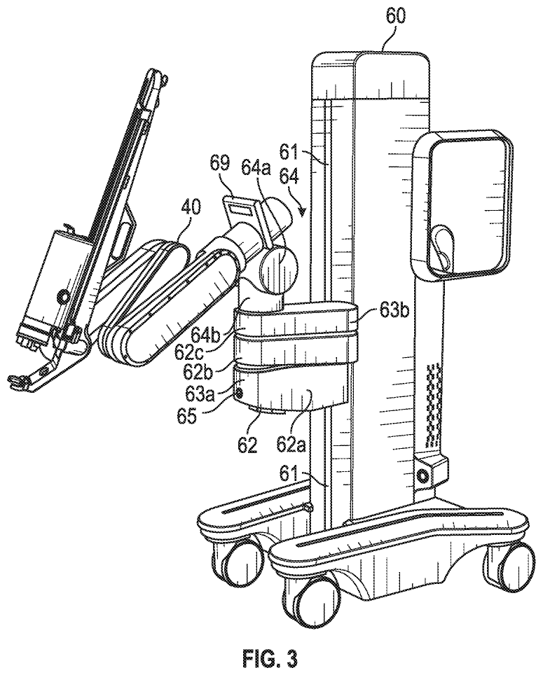

With reference to FIGS. 2 and 3, each of the robotic arms 40 may include a plurality of links 42a, 42b, 42c, which are interconnected at joints 44a, 44b, 44c, respectively. Joint 44a is configured to secure the robotic arm 40 to the movable cart 60 and defines a first longitudinal axis. The movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62a and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIGS. 1 and 2, the robotic arm 40 also includes a holder 46 defining a second longitudinal axis and configured to receive an IDU 52. The IDU 52 is configured to couple to an actuation mechanism of the surgical instrument 50 and the endoscope camera 51 and is configured to move (e.g., rotate) and actuate the instrument 50 and/or the endoscope camera 51. IDU 52 transfers actuation forces from its actuators to the surgical instrument 50 and/or the endoscope camera 51to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 further includes a plurality of manual override buttons 53 disposed on the IDU 52 and/or the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a remote center point “P” which lies at an intersection of the first axis defined by the link 42a and the second axis defined by the holder 46. Thus, the actuator 48b controls the angle “A” between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle “A.” In aspects, some or all of the joints 44a, 44b, 44c may include an actuator to obviate the need for mechanical linkages.

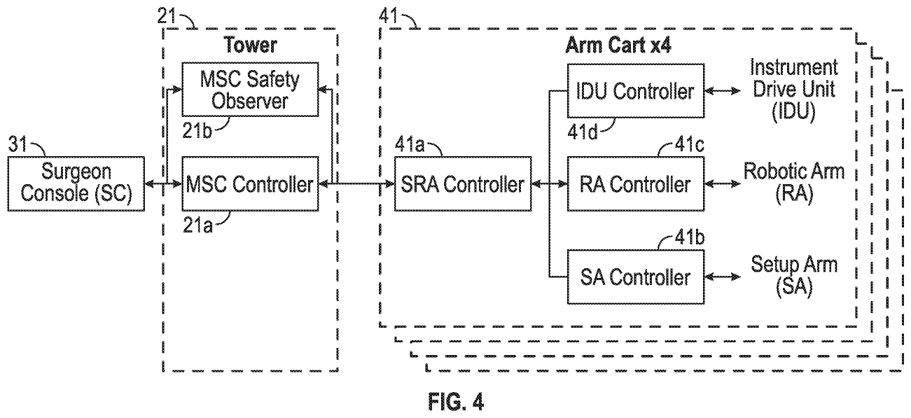

With reference to FIGS. 1 and 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The handle controllers 38a and 38b include one or more haptic feedback vibratory devices that output haptic feedback. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an IDU controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

With additional reference to FIGS. 2 and 3, the setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

Turning to FIGS. 5-7, a surgical instrument 110 provided in accordance with the present disclosure generally includes a housing 120, a shaft 130 extending distally from housing 120, an end effector assembly 140 extending distally from shaft 130, and an actuation assembly 1100 disposed within housing 120 and operably associated with end effector assembly 140. Instrument 110 is detailed herein as an articulating electrosurgical forceps configured for use with a surgical robotic system, e.g., surgical robotic system 10 (FIG. 1). However, the aspects and features of instrument 110 provided in accordance with the present disclosure, detailed below, are equally applicable for use with other suitable surgical instruments, e.g., graspers, staplers, clip appliers, and/or in other suitable surgical systems, e.g., motorized, other power driven systems, and/or manually actuated surgical systems (including handheld instruments).

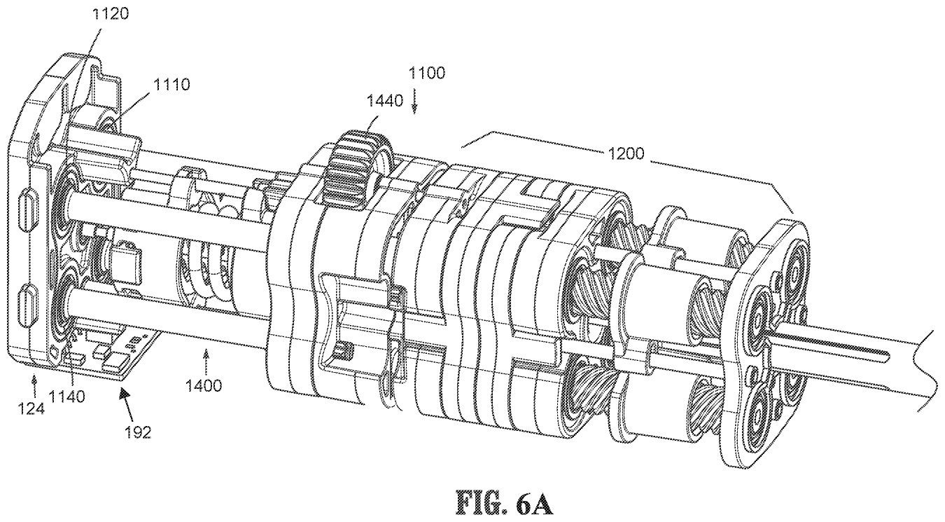

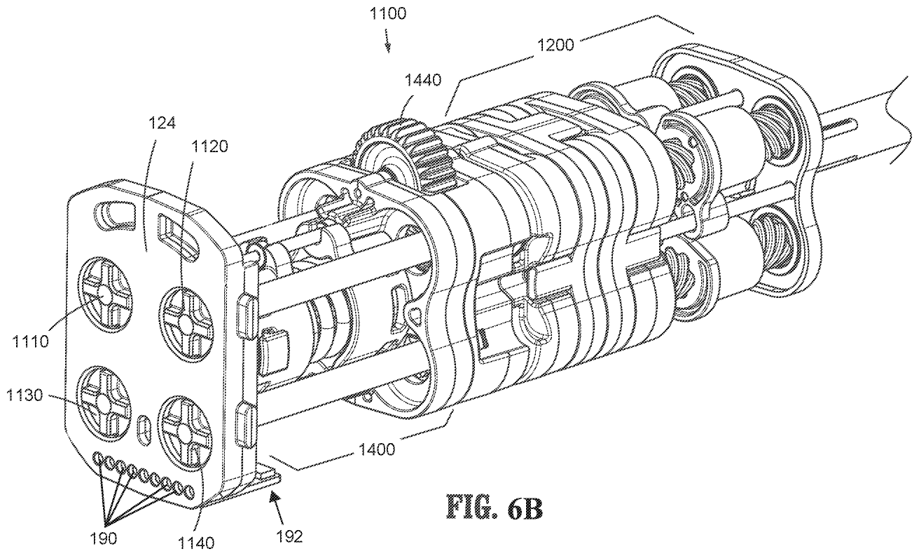

With particular reference to FIG. 5, housing 120 of instrument 110 includes first and second housing parts 122a, 122b and a proximal face plate 124 that cooperate to enclose actuation assembly 1100 therein. Proximal face plate 124 includes through holes defined therein through which input couplers 1110-1140 (FIG. 6B) of actuation assembly 1100 extend. A pair of latch levers 126 (only one of which is illustrated in FIG. 5) extending outwardly from opposing sides of housing 120 enable releasable engagement of housing 120 with a robotic arm 40 (FIG. 1) of a surgical robotic system, e.g., surgical robotic system 10 (FIG. 1). A window 128 defined through housing 120 permits thumbwheel 1440 to extend therethrough to enable manual manipulation of thumbwheel 1440 from the exterior of housing 120 to permit manual opening and closing of end effector assembly 140.

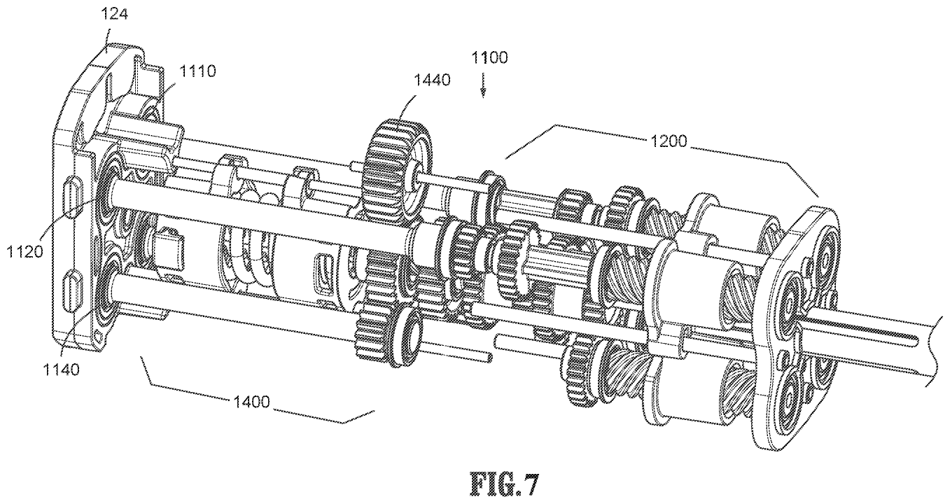

Referring also to FIGS. 6A-7, a plurality of electrical contacts 190 extend through one or more apertures defined through proximal face plate 124 to enable electrical communication between instrument 110 and surgical robotic system 10 (FIG. 1) when instrument 110 is engaged on a robotic arm thereof, e.g., for the communication of data, control, and/or power signals therebetween. As an alternative to electrical contacts 190 extending through proximal face plate 124, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 190 are electrically coupled with electronics 192 mounted on an interior side of proximal face plate 124, e.g., within housing 120. Electronics 192 may include, for example, a storage device, a communications device (including suitable input/output components), and a CPU including a memory and a processor. Electronics 192 may be mounted on a circuit board or otherwise configured, e.g., as a chip.

The storage device of electronics 192 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 192 may be, for example, a magnetic disk, flash memory, optical disk, or other suitable data storage device.

As an alternative or in addition to storing the above noted information in the storage device of electronics 192, some or all of such information, e.g., the use information, calibration information, setting information, and/or adjustment information, may be stored in a storage device associated with surgical robotic system 10 (FIG. 1), a remote server, a cloud server, etc., and accessible via instrument 110 and/or surgical robotic system 10 (FIG. 1). In such configurations, the information may, for example, be updated by manufacturer provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer provided updates manually or automatically upon connection to the surgical robotic system 10 (FIG. 1).

Referring again to FIG. 5, shaft 130 of instrument 110 includes a distal clevis segment 132, a proximal segment 134, and an articulating section 136 disposed between the distal clevis and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal clevis segment 132 of shaft 130 at the distal ends thereof and extend proximally from distal clevis segment 132 of shaft 130, through articulating section 136 of shaft 130 and proximal segment 134 of shaft 130, and into housing 120, wherein articulation cables 138 operably couple with an articulation sub-assembly 1200 of actuation assembly 1100 (FIG. 6A) to enable selective articulation of distal clevis segment 132 (and, thus end effector assembly 140) relative to proximal segment 134 and housing 120, e.g., about at least two axes of articulation (yaw and pitch articulation, for example). Articulation cables 138 are arranged in a generally rectangular configuration, although other suitable configurations are also contemplated. In some configurations, as an alternative, shaft 130 is substantially rigid, malleable, or flexible and not configured for active articulation. Articulation sub-assembly 1200 is described in greater detail below.

With respect to articulation of end effector assembly 140 relative to proximal segment 134 of shaft 130, actuation of articulation cables 138 may be accomplished in pairs. More specifically, in order to pitch end effector assembly 140, the upper pair of cables 138 are actuated in a similar manner while the lower pair of cables 138 are actuated in a similar manner relative to one another but an opposite manner relative to the upper pair of cables 138. With respect to yaw articulation, the right pair of cables 138 are actuated in a similar manner while the left pair of cables 138 are actuated in a similar manner relative to one another but an opposite manner relative to the right pair of cables 138. Other configurations of articulation cables 138 or other articulation actuators are also contemplated.

Continuing with reference to FIG. 5, end effector assembly 140 includes first and second jaw members 142, 144, respectively. Each jaw member 142, 144 includes a proximal flange 143a, 145a and a distal body 143b, 145b, respectively. Distal bodies 143b, 145b define opposed tissue contacting surfaces 146, 148, respectively. Proximal flanges 143a, 145a are pivotably coupled to one another about a pivot 150 and are operably coupled to one another via a cam slot assembly 152 including a cam pin slidably received within cam slots defined within the proximal flange 143a, 145a of at least one of the jaw members 142, 144, respectively, to enable pivoting of jaw member 142 relative to jaw member 144 and distal segment 132 of shaft 130 between a spaced apart position (e.g., an open position of end effector assembly 140) and an approximated position (e.g., a closed position of end effector assembly 140) for grasping tissue between tissue contacting surfaces 146, 148. As an alternative to this unilateral configuration, a bilateral configuration may be provided whereby both jaw members 142, 144 are pivotable relative to one another and distal segment 132 of shaft 130. Alternatively, the above detailed configuration may be reversed, e.g., wherein jaw member 142 is the fixed jaw member and jaw member 144 is movable relative to jaw member 142. Other suitable jaw actuation mechanisms (for bilateral and/or unilateral jaw configurations) are also contemplated.

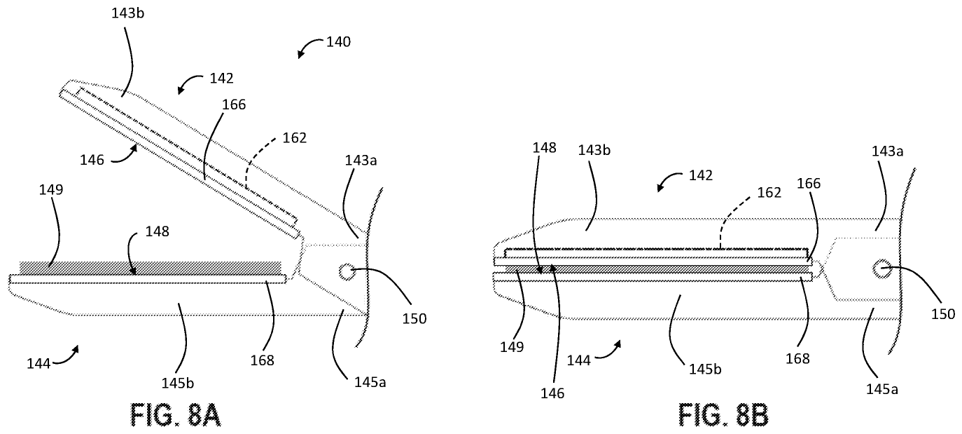

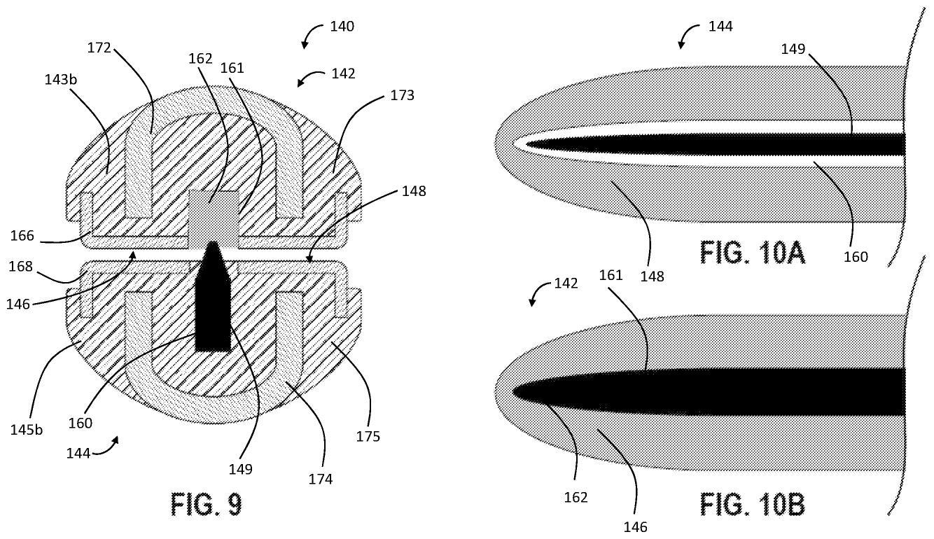

In configurations, jaw member 144 supports a longitudinally extending cutting electrode 149 in a slot 160 defined through tissue contacting surface 148 and a portion of distal body 145b of jaw member 144, while jaw member 142 includes a compression pad 162 (FIGS. 8A-9) disposed in a slot 161 (FIG. 9) defined through tissue contacting surface 146 and a portion of distal body 143b of jaw member 142. In such aspects, in the approximated position of jaw members 142, 144, cutting electrode 149 is urged into contact with compression pad 162 (FIGS. 8A-9) to grasp (and, in aspects, tension) tissue therebetween. Cutting electrode 149 may then be energized to cut the tissue disposed between cutting electrode 149 and compression pad 162 (FIGS. 8A-9). Cutting electrode 149 may additionally or alternatively be used to cut tissue in an open jaw configuration, e.g., with jaw members 142, 144 disposed in the spaced apart position. Cutting electrode 149 may be configured to be energized with monopolar Radio Frequency (RF) energy from a surgical generator (not shown) to conduct RF energy to tissue to cut the tissue, wherein the RF energy is returned to the generator to complete the circuit via a remote return device such as a return pad (not shown) or a local return device such as another portion of end effector assembly 140 or a separate instrument (not shown), e.g., a tenaculum, a probe, etc. Alternatively or additionally, cutting electrode 149 may be energized with bipolar RF energy wherein energy conducted from cutting electrode 149 to tissue is returned via either or both of tissue contacting surfaces 146, 148 of jaw members 142, 144, respectively, or other suitable local return device.

Referring still to FIG. 5, a drive rod 1484 is operably coupled to cam slot assembly 152 of end effector assembly 140, e.g., engaged with the cam pin thereof, such that longitudinal actuation of drive rod 1484 pivots jaw member 142 relative to jaw member 144 between the spaced apart and approximated positions. More specifically, urging drive rod 1484 proximally pivots jaw member 142 relative to jaw member 144 towards the approximated position while urging drive rod 1484 distally pivots jaw member 142 relative to jaw member 144 towards the spaced apart position. However, other suitable mechanisms and/or configurations for pivoting jaw member 142 relative to jaw member 144 between the spaced apart and approximated positions in response to selective actuation of drive rod 1484 are also contemplated. Drive rod 1484 extends proximally from end effector assembly 140 through shaft 130 and into housing 120 wherein drive rod 1484 is operably coupled with a jaw drive sub-assembly 1400 of actuation assembly 1100 (FIGS. 6A-6B) to enable selective actuation of end effector assembly 140 to grasp tissue therebetween and apply a jaw force within an appropriate jaw force range.

Tissue contacting surfaces 146, 148 of jaw members 142, 144, respectively, are at least partially formed from an electrically conductive material and are energizable to different potentials to enable the conduction of RF electrical energy through tissue grasped therebetween, although tissue contacting surfaces 146, 148 may alternatively be configured to supply any suitable energy, e.g., thermal, microwave, light, ultrasonic, etc., through tissue grasped therebetween for energy based tissue treatment. Instrument 110 defines a conductive pathway (not shown) through housing 120 and shaft 130 to end effector assembly 140 that may include lead wires, contacts, and/or electrically conductive components to enable electrical connection of tissue contacting surfaces 146, 148 of jaw members 142, 144, respectively, to an energy source (not shown), e.g., an electrosurgical generator, for supplying energy to tissue contacting surfaces 146, 148 to treat, e.g., seal, tissue grasped between tissue contacting surfaces 146, 148.

With additional reference to FIGS. 6A-7, as noted above, actuation assembly 1100 is disposed within housing 120 and includes an articulation sub-assembly 1200, and a jaw drive sub-assembly 1400. Articulation sub-assembly 1200 is operably coupled between first and second input couplers 1110, 1120, respectively, of actuation assembly 1100 and articulation cables 138 (FIG. 5) such that, upon receipt of appropriate inputs into first and/or second input couplers 1110, 1120, articulation sub-assembly 1200 manipulates cables 138 (FIG. 5) to articulate end effector assembly 140 in a desired direction, e.g., to pitch and/or yaw end effector assembly 140. Articulation sub-assembly 1200 is described in greater detail below.

Jaw drive sub-assembly 1400 is operably coupled between fourth input coupler 1140 of actuation assembly 1100 and drive rod 1484 such that, upon receipt of appropriate input into fourth input coupler 1140, jaw drive sub-assembly 1400 pivots jaw members 142, 144 between the spaced apart and approximated positions to grasp tissue therebetween and apply a jaw force within an appropriate jaw force range.

Actuation assembly 1100 is configured to operably interface with a surgical robotic system, e.g., system 10 (FIG. 1), when instrument 110 is mounted on a robotic arm thereof, to enable robotic operation of actuation assembly 1100 to provide the above detailed functionality. That is, surgical robotic system 10 (FIG. 1) selectively provides inputs, e.g., rotational inputs to input couplers 1110-1140 of actuation assembly 1100 to articulate end effector assembly 140, grasp tissue between jaw members 142, 144, and/or cut tissue grasped between jaw members 142, 144. However, as noted above, it is also contemplated that actuation assembly 1100 be configured to interface with any other suitable surgical systems, e.g., a manual surgical handle, a powered surgical handle, etc.

Turning to FIGS. 8A-10B, a distal portion of end effector assembly 140 is shown with jaw members 142, 144 disposed in the spaced apart position (FIG. 8A) and the approximated position (FIGS. 8B and 9). In aspects, either or both tissue contacting surfaces 146, 148 of jaw members 142, 144, respectively, are defined by respective tissue contacting plates 166, 168 disposed on the opposing surfaces of distal bodies 143b, 145b of jaw members 142, 144, respectively. As detailed above, jaw member 144 supports cutting electrode 149 in slot 160 defined through tissue contacting surface 148 (and through tissue contacting plate 168 and a portion of distal body 145b of jaw member 144), while jaw member 142 includes compression pad 162 disposed in slot 161 defined through tissue contacting surface 146 (and through tissue contacting plate 166 and a portion of distal body 143b of jaw member 142) and configured to oppose cutting electrode 149 in the approximated position of jaw members 142, 144 (FIGS. 8B and 9). Tissue contacting surfaces 146, 148 may define substantially U-shaped configurations wherein the slots 161, 160 defined therethrough terminate at positions proximally spaced from the distal ends of tissue contact surfaces 146, 148 (see FIGS. 10A and 10B).

Cutting electrode 149 protrudes from jaw member 144 beyond tissue contacting plate 168 and towards jaw member 142. Compression pad 162 may be substantially flush with tissue contacting surface 146 of tissue contacting plate 166, may be recessed relative thereto, or may protrude from tissue contacting surface 146 of tissue contacting plate 166 towards jaw member 144. Compression pad 162 and jaw member 142 are configured, in conjunction with cutting electrode 149, such that, in the approximated position of jaw members 142, 144 (see FIGS. 8B and 9), cutting electrode 149 is urged into and at least partially compresses compression pad 162 (with tissue grasped therebetween), thus facilitating electrical tissue cutting upon activation of cutting electrode 149. The contact between cutting electrode 149 and compression pad 162 may also maintain a spacing between tissue contacting surfaces 146, 148 to inhibit electrical shorting via contact therebetween.

Either or both jaw members 142, 144 may include a structural jaw support 172, 174 defining the respective proximal flange 143a, 145a of the jaw member 142, 144 and extending into the respective distal body 143b, 145b. In such configurations, distal body 143b, 145b of either or both jaw members 142, 144 may further include jaw housings 173, 175 surrounding structural jaw supports 172, 174 and supporting tissue contacting plates 166, 168, respectively, thereon. Jaw housings 173, 175 may be formed from insulative materials and, in aspects, may be overmolded about jaw supports 172, 174 and a portion of tissue contacting plates 166, 168 to form jaw members 142, 144 and secure the components thereof to one another. In other configurations, jaw housings 173, 175 are conductive and electrically isolated from the other components of jaw members 142, 144 via suitable insulation. Alternatively or additionally, either or both jaw members 142, 144 may be formed from a monolithic, electrically conductive piece of material defining the structural jaw support, tissue contacting surface, and jaw housing thereof. At least a portion of the jaw housing, in such configurations, may be coated with an insulative material. Further, with respect to configurations where jaw member 144 is formed from a monolithic piece of material, cutting electrode 149 may be electrically isolated from the remainder of jaw member 144, e.g., via an insulator disposed therebetween. Thus, as utilized herein, reference to jaw housings 173, 175 includes insulative jaw housings, conductive jaw housings, and/or monolithic jaw structures defining jaw housings. Further, both jaw members 142, 144 may be similarly configured or may define different configurations, such as any combination of the jaw configurations detailed herein.

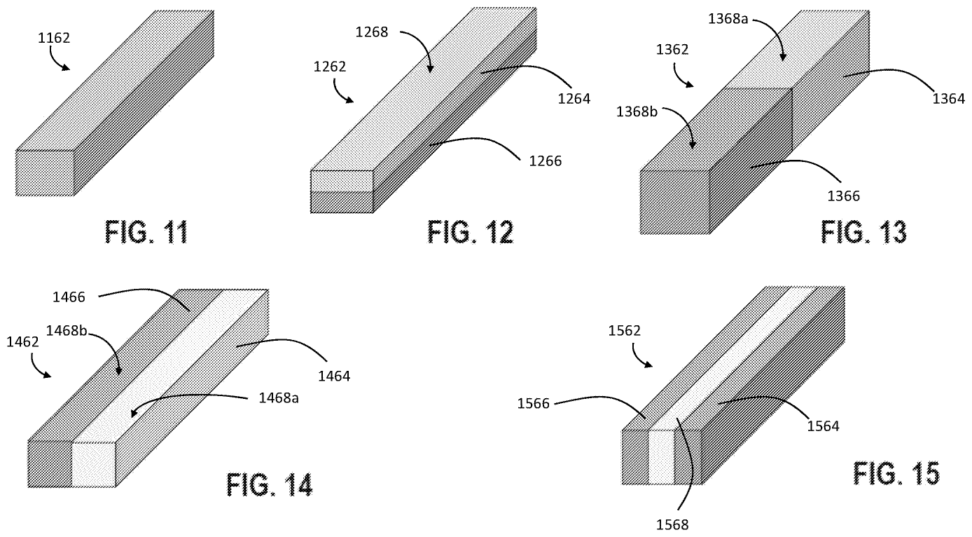

Turning to FIGS. 11-30, in conjunction with FIGS. 8-10B, as noted above, compression pad 162 and jaw member 142 are configured to facilitate electrical cutting of tissue grasped between jaw members 142, 144 upon activation of cutting electrode 149. More specifically, compression pad 162 is at least partially resiliently compressible and defines a suitable durometer, suitable durometer profile (e.g., with portions having different durometers), suitable size and shape configuration, and/or suitable configuration relative to jaw member 142 (and jaw member 142 may define a suitable configuration in conjunction with compression pad 162), to facilitate grasping tissue between compression pad 162 and cutting electrode 149 with sufficient force (and, in aspects, suitable tension) to enable effective and efficient electrical cutting of tissue upon activation of cutting electrode 149. Various aspects and features of compression pad 162 and/or jaw member 142 to enable this effective and efficient electrical cutting of tissue upon activation of cutting electrode 149 are detailed below with reference to FIGS. 11-30. To the extent consistent, any or all of these aspects and features may be used in any suitable combination with any or all of the other aspects and features. Further, although some of the compression pads detailed below are shown isolated and not disposed within jaw member 142 to better illustrate the features thereof, it is understood that these compression pads are configured for positioning within jaw member 142 in any of the manners detailed herein. Further, orientational terms (e.g., proximal, distal, upper, lower, etc.) referenced with respect to these compression pads correspond to the same orientations of jaw member 142.

As shown in FIG. 10B, compression pad 162 may taper in width along at least a portion of the length of compression pad 162 in a proximal to distal direction along jaw member 142. The width taper of compression pad 162 may correspond with a similar width taper of jaw member 142. Alternatively, compression pad 162 may taper in width differently from a taper in width of jaw member 142, or may taper in width without jaw member 142 also tapering in width. In aspects, the taper in width of compression pad 162 is uniform, non-uniform, continuous, stepped, random, or provided in any other suitable manner. The width of slot 161 may likewise taper with the width of compression pad 162 or may taper (or not taper) differently from compression pad 162.

Regardless of the particular configuration of the taper of compression pad 162, the taper in width of compression pad 162 facilitates providing a substantially uniform, or more uniform, pressure applied to tissue grasped between compression pad 162 and cutting electrode 149 (FIG. 9). More specifically, given that the force applied to tissue grasped between compression pad 162 and cutting electrode 149 (FIG. 9) decreases with an increase in distance from the pivot, e.g., pivot 150, the force applied is greater towards the proximal end of compression pad 162 compared to the force applied towards the distal end of compression pad 162. To at least partially counteract this decrease in force applied in the proximal to distal direction, the taper in width of compression pad 162 reduces the exposed surface area of compression pad 162 in a proximal to distal direction along jaw member 142. Thus, while the force applied decreases in the proximal to distal direction, the surface area also decreases in the proximal to distal direction such that the pressure (defined as force per unit of surface area) applied to tissue grasped between compression pad 162 and cutting electrode 149 (FIG. 9) is substantially uniform, or more uniform, along the length of jaw member 142. In other aspects, compression pad 162 does not taper in width or defines other suitable tapering portion(s).

Referring to FIG. 11, a compression pad configured for use with jaw member 142 (FIGS. 8A-9 and 10B) or any other suitable jaw member is shown identified by reference numeral 1162. Although compression pad 1162 is illustrated defining a rectangular prism shaped configuration, compression pad 1162 may define any other suitable shape configuration, such as but not limited to those detailed herein.

Compression pad 1162 is substantially uniform in shape and/or material(s) along the length of compression pad 1162, across the width of compression pad 1162, and/or through the depth of compression pad 1162 such that compression pad 1162 exhibits substantially similar properties, e.g., durometer, across these dimension(s). Compression pad 1162 may be substantially solid, e.g., without openings therethrough or therein, and may be formed from any suitable material such as, for example, silicone or polytetrafluoroethylene (PTFE). Other suitable resiliently compressible materials having sufficient thermal properties are also contemplated for forming at least a portion of compression pad 1162 such as, for example, resiliently compressible materials capable of withstanding temperatures of, in aspects, at least 200° C.; in other aspects, of at least 240° C.; or, in still other aspects, of at least 260° C. In aspects, compression pad 1162 is formed from an overmold or injection moldable material or materials.

Compression pad 1162, in aspects, may be formed from a single material or a substantially homogeneous mixture of materials. Alternatively or additionally, compression pad may include filler materials disposed thereon (e.g., on the tissue contacting surface thereof) or therein (e.g., uniformly or non-uniformly distributed throughout compression pad 1162). Such filler materials include, without limitation: calcium carbonate, talc, silica, wollastonite, clay, calcium sulfate fibers, mica, glass beads, and alumina trihydrate. Filler materials such as those noted above provide texture and/or roughness which increases gripping and reduces slippage of tissue grasped between compression pad 162 and cutting electrode 149 (FIG. 9). Such filler materials may also increase the effective durometer of compression pad 162, at least in the portions of compression pad 162 where such filler materials are provided.

Turning to FIGS. 12-15, compression pads 1262, 1362, 1462, 1562 are shown including variable durometers and/or other properties across at least one dimension thereof. Compression pads 1262, 1362, 1462, 1562 are shown defining rectangular prism shaped configurations, although any other suitable shape configurations are also contemplated. In order to vary the durometers and/or other properties across compression pads 1262, 1362, 1462, 1562, compression pads 1262, 1362, 1462, 1562 may include different materials forming different portions thereof, as detailed below.

As shown in FIG. 12, compression pad 1262 includes a first compression pad portion 1264 and a second compression pad portion 1266. First and second compression pad portions 1264, 1266 are stacked atop one another with first compression pad portion 1264 defining a tissue contacting surface 1268 of compression pad 1262 while second compression pad portion 1266 is configured to be at least partially recessed within jaw member 142 (FIG. 9) and, thus, at least partially unexposed. First and second compression pad portions 1264, 1266 of compression pad 1262 may define different durometers. For example, first and second compression pad portions 1264, 1266 of compression pad 1262 may both be resiliently compressible elastomers with different durometers such that, upon compression of compression pad 1262 in a height direction of compression pad 1262, one of first or second compression pad portions 1264, 1266 is at least partially compressed prior to the compression of the other of the first and second compression pad portions 1264, 1266 and/or more than the other of the first and second compression pad portions 1264, 1266. In other aspects, one of first and second compression pad portions 1264, 1266 is a resiliently compressible elastomer while the other of first and second compression pad portions 1264, 1266 substantially resists compression during normal use conditions.

Alternatively or additionally, first and second compression pad portions 1264, 1266 may differ in thermal conductive, e.g., wherein one of first and second compression pad portions 1264, 1266 is thermally conductive while the other of first and second compression pad portions 1264, 1266 is a thermal insulator. Likewise, one of first and second compression pad portions 1264, 1266 may include a filler material, while the other of first and second compression pad portions 1264, 1266 is devoid of a filler material, includes a different filler material, or a different amount (by weight or volume) of filler material.

With reference to FIG. 13, compression pad 1362 includes a first, proximal compression pad portion 1364 and a second, distal compression pad portion 1366. First and second compression pad portions 1364, 1366 are aligned with one another in a proximal to distal direction with first compression pad portion 1364 defining a proximal tissue contacting surface 1368a of compression pad 1362 while second compression pad portion 1366 defines a distal tissue contacting surface 1368b of compression pad 1362. First and second compression pad portions 1364, 1366 of compression pad 1362 may differ from one another in any of the manners detailed above with respect to first and second compression pad portions 1264, 1266 of compression pad 1262 (FIG. 12). In one particular implementation, first and second compression pad portions 1364, 1366 are both resiliently compressible elastomers with different durometers. For example, first, proximal compression pad portion 1364 may define a first durometer while second, distal compression pad portion 1366 defines a second, greater durometer such that, upon closure of jaw members 142, 144 to grasp tissue between compression pad 1362 and cutting electrode 149 (see FIG. 9), first, proximal compression pad portion 1364 compresses a relatively greater amount than second, distal compression pad portion 1366, thus facilitating substantially uniform, or more uniform, force applied to tissue along the length compression pad 1362 and/or a substantially uniform, or more uniform, gap distance defined between tissue contacting surfaces 146, 148 (FIG. 9) along the length compression pad 1362 given that forces may be greater, and/or gap distance smaller, at locations closer to pivot 150 (FIGS. 8A and 8B) as compared to locations farther from pivot 150 (FIGS. 8A and 8B).

Referring to FIG. 14, compression pad 1462 includes a first, right compression pad portion 1464 and a second, left compression pad portion 1466. First and second compression pad portions 1464, 1466 are positioned side by side with one another across jaw member 142 (FIG. 9) with first compression pad portion 1464 defining a right tissue contacting surface 1468a of compression pad 1462 while second compression pad portion 1466 defines a left tissue contacting surface 1468b of compression pad 1462. First and second compression pad portions 1464, 1466 of compression pad 1462 may differ from one another in any of the manners detailed above with respect to first and second compression pad portions 1264, 1266 of compression pad 1262 (FIG. 12). In one particular implementation, first and second compression pad portions 1464, 1466 are both resiliently compressible elastomers with different durometers. For example, first compression pad portion 1464 may define a first durometer while second compression pad portion 1466 defines a second, different durometer. In aspects, compression pad 1462 is configured for use with laterally curved jaw members wherein first compression pad portion 1464 is disposed towards the inner or concave edge of the curved jaw members while second compression pad portion 1466 is disposed towards the outer or convex edge of the curved jaw members. In such configurations, first compression pad portion 1464 may define a first durometer less than a second durometer of second compression pad portion 1466, thus facilitating substantially uniform, or more uniform, force applied to tissue across the width of compression pad 1462, e.g., given that forces may tend to be greater towards the inner or concave edge of the curved jaw members compared to the outer or convex edge of the curved jaw members.

FIG. 15 illustrates compression pad 1562 including a first, right compression pad portion 1564, a second, left compression pad portion 1566, and a third, center compression pad portion 1568. First, second, and third, compression pad portions 1564, 1566, 1568 are positioned side by side with one another across jaw member 142 (FIG. 9). First and second compression pad portions 1564, 1566 of compression pad 1562 may be similar to one another, while third compression pad portion 1568 differs from first and second compression pad portions 1564, 1566 in any of the manners detailed above with respect compression pad 1262 (FIG. 12). In one particular implementation, first second, and third compression pad portions 1564, 1566 are all resiliently compressible elastomers with first and second compression pad portions 1564, 1566 defining a first durometer while third compression pad portion 1568 defines a second, different durometer. The second, different durometer may be greater than the first durometer to increase force at the lateral center of compression pad 1562 or may be less than the first durometer to facilitate a uniform or more uniform force profile laterally across compression pad 1562. In aspects, third compression pad portion 1568 defines a width substantially equal to or greater than a width of cutting electrode 149 (FIG. 9) such that cutting electrode 149 (FIG. 9) is urged into third compression pad portion 1568 in the approximated position of jaw members 142, 144 (FIG. 9). First and second compression pad portions 1564, 1566 may define equal or different widths as compared to third compression pad portion 1568. In other aspects, first and second compression pad portions 1564, 1566 each define a thermal conductance (similar or different) that is less than a thermal conductance of third compression pad portion 1568. This configuration may facilitate concentrating heat at the center of compression pad 1562, e.g., opposing cutting electrode 149 (FIG. 9), to facilitate heating and, thus, cutting of tissue.

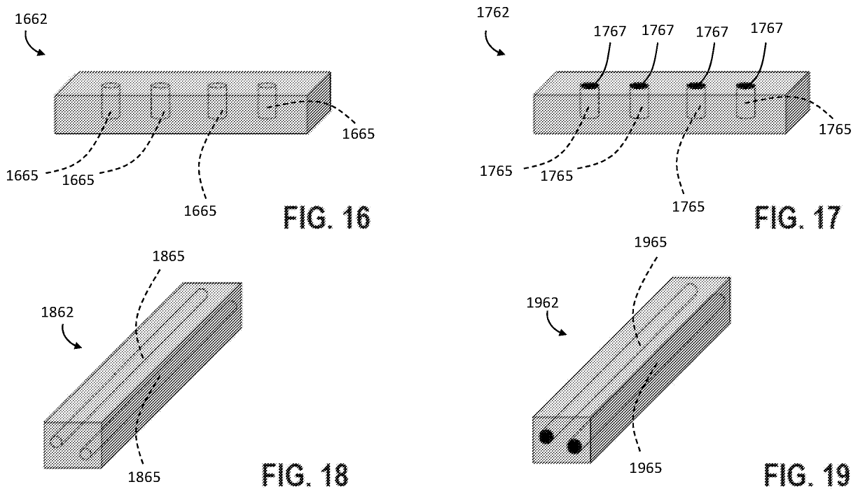

Turning to FIGS. 16-19, compression pads 1662, 1762, 1862, 1962 are shown including interruptions designed to vary the durometer and/or other properties across at least one dimension thereof. Compression pads 1662, 1762, 1862, 1962 are shown defining rectangular prism shaped configurations, although any other suitable shape configurations are also contemplated.

Referring to FIG. 16, in order to vary the durometer (and/or other properties) across at least one dimension of compression pad 1662, compression pad 1662 includes a plurality of hollow voids 1665 defined at least partially through a height or thickness of compression pad 1662. Hollow voids 1665 may be spaced-apart from one another and aligned to define one or more longitudinal rows extending proximally to distally along at least a portion of a length of compression pad 1662 (as shown), may be spaced-apart from one another and aligned to define one or more lateral rows extending laterally across at least a portion of a width of compression pad, may be defined towards one side of compression pad 1662, may be defined towards one end of compression pad 1662, may be defined down a center or along the sides of compression pad 1662, and/or may be defined in any other suitable manner. Hollow voids 1665 increase the compressibility of compression pad 1662 at least in the vicinity of each hollow void 1665 and, thus, hollow voids 1665 can be used to increase compressibility of compression pad 1662 (lowering the effective durometer of compression pad 1662) at certain locations or portions of compression pad 1662.

With reference to FIG. 17, in aspects, in order to vary the durometer (and/or other properties) across at least one dimension of compression pad 1762, compression pad 1762 includes a plurality of filled voids 1765 defined at least partially through a height or thickness of compression pad 1762 and at least partially filled with a filler 1767 different from the material forming the body of compression pad 1762. Filled voids 1765 may be arranged in any of the configurations detailed above with respect to compression pad 1662 (FIG. 16) or in any other suitable configuration.

Filler 1767 may define a durometer greater than the body of compression pad 1762 to define areas of reduced compression, or may define a durometer less than the body of compression pad 1762 to define areas of increased compression. Alternatively or additionally, filler 1767 may define a texture different from the texture of the body of compression pad 1762 to provide greater or less tissue grasping texture in certain areas of compression pad 1762 and/or may define a thermal conductance different from the thermal conductance of the body of compression pad 1762 to provide greater or less thermal conductance in certain areas of compression pad 1762.

FIGS. 18 and 19 illustrate compression pads 1862, 1962 including hollow voids 1865 and filled voids 1965 defined at least partially through a length of compression pads 1862, 1962, respectively. Other than the direction of hollow voids 1865 and filled voids 1965, compression pads 1862, 1962 may otherwise be configured in accordance with any of the aspects of respective compression pads 1662, 1762 (FIGS. 16 and 17), detailed above. It is alternatively or additionally contemplated that hollow and/or filled voids may be defined at least partially through a width of a compression pads in a similar manner as detailed above with respect to compression pads 1662, 1762 (FIGS. 16 and 17).

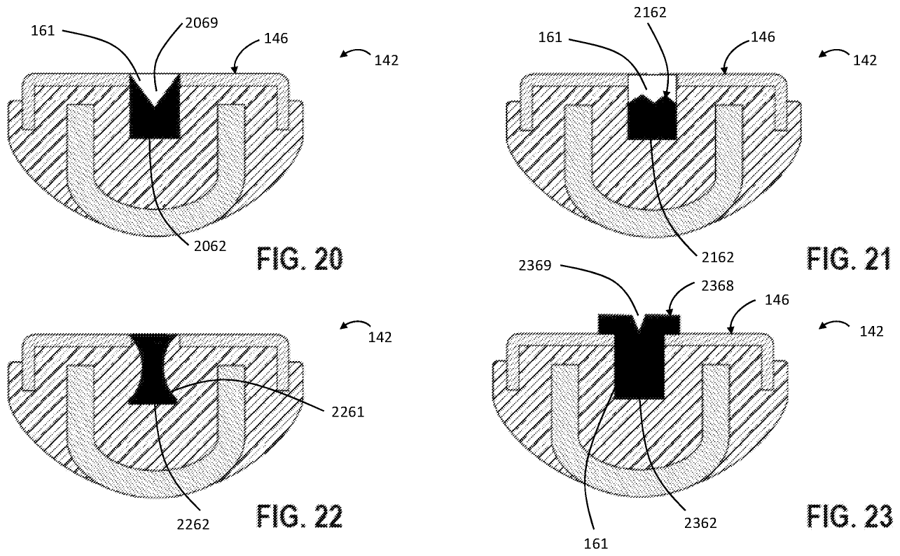

Turning to FIGS. 20-27, in accordance with this disclosure, the compression pad may itself be configured and/or configured relative to jaw member 142 to provide a suitable force or force profile along and/or across jaw member 142. For example, as shown in FIG. 20, compression pad 2062 is disposed within slot 161 of jaw member 142. More specifically, compression pad 2062 substantially fills the entirety of slot 161 and extends to a substantially coplanar extent relative to tissue contacting surface 146, except that compression pad 2062 defines a cutout 2069 such that a portion of compression pad 2062 is recessed within slot 161 relative to tissue contacting surface 146. Cutout 2069 may be laterally centered within compression pad 2062 such that lateral side portions of compression pad 2062 extend to substantially coplanar relation with tissue contacting surface 146 while a center portion of compression pad 2062 remains recessed relative to tissue contacting surface 146. This configuration may at least partially account for cutting electrode 149 (FIG. 9), allowing cutting electrode 149 to at least partially extend into cutout 2069 in the approximated position of jaw members 142, 144 (see FIG. 9). However, other configurations of cutouts 2069 defined within compression pad 2062 are also contemplated. Compression pad 2062 may define portions of different durometer and/or other properties similar to any of the aspects described above.

Referring to FIG. 21, compression pad 2162 is disposed within slot 161 of jaw member 142 and is fully recessed within slot 161 and relative to tissue contacting surface 146. Compression pad 2162 may define a substantially planar exposed surface 2163 or, as shown, may define a varied profile across a width dimension of exposed surface 2163 such as, for example, a triangle wave configuration (as shown), a sine wave configuration, a square wave configuration, one or more protrusions and/or recesses, a convex or concave exposed surface 2163 or portion thereof, etc. Compression pad 2162 may define portions of different durometer and/or other properties similar to any of the aspects described above.

With reference to FIG. 22, slot 2261 of jaw member 142 defines a varied width along a height dimension thereof such as, for example, defining an hourglass configuration (as shown), although other configurations are also contemplated. Compression pad 2262 substantially fills the entirety of slot 2261 and may define a complementary shape as slot 2261 or may assume the shape of slot 2261 as a result of compression of compression pad 2262 during installation into or formation within slot 2261. The reduced width in compression pad 2262, and the replacement thereof with the substantially rigid distal body 143b of jaw member 142 at the reduced width portions of slot 2261 decrease the compressibility of compression pad 2262 at least in the reduced-width areas, thus increases the effective durometer over at least a portion of compression pad 2262. Compression pad 2262 may also define portions of different durometer and/or other properties similar to any of the aspects described above.

FIG. 23 shows compression pad 2362 is disposed within slot 161 of jaw member 142 and protruding from slot 161 towards jaw member 142 (FIG. 9). Further, this protruding portion of compression pad 2362 also extends laterally outwardly to partially overlap tissue contacting surface 146 on either side of slot 161. Compression pad 2362 further defines, in some aspects, a cutout 2369 through a tissue contacting surface 2368 thereof, although additional or alternative features of tissue contacting surface 2368 are also contemplated. Compression pad 2362 may also define portions of different durometer and/or other properties similar to any of the aspects described above.

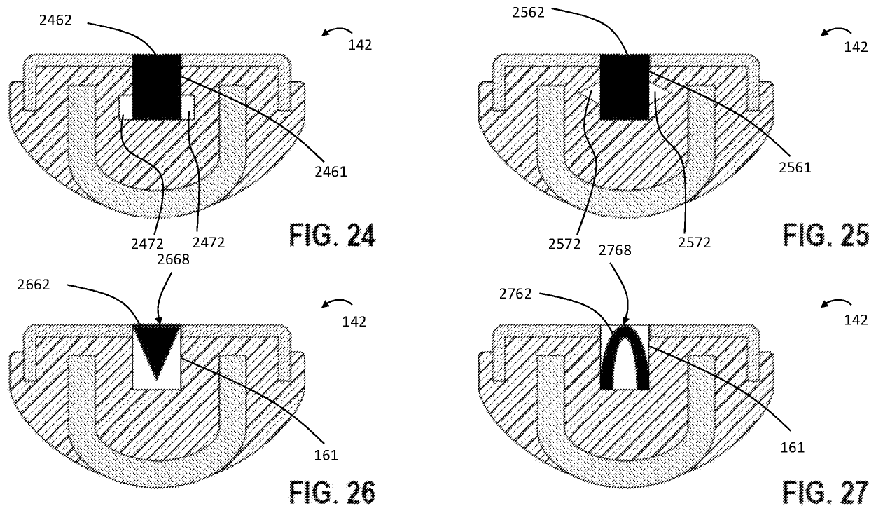

Referring to FIGS. 24 and 25, in aspects, a slot 2461, 2561 of jaw member 142 defines relief recesses 2472, 2572 within opposing side walls defining the slot 2461, 2561. Relief recesses 2472, 2572 may extend the entirety or a portion of a length of the slot 2461, 2561 and may define any suitable configuration such as, for example, rectangular (FIG. 24), triangular (FIG. 25), radiused, etc. Further, the relief recesses 2472, 2572 may be disposed towards the closed end of the slot 2461, 2561 (see FIG. 24), at an intermediate position along the height of the slot 2461, 2561 (see FIG. 5), at the open end of the slot 2461, 2561, or at any other suitable position. Multiple relief recesses 2472, 2572 are also contemplated. In an initial, at-rest condition of the compression pad 2462, 2562, the relief recesses 2472, 2572 are substantially unfilled. However, upon compression of the compression pad 2462, 2562, the compression pad 2462, 2562 is urged at least partially into the relief recesses 2472, 2572. Thus, by providing the relief recesses 2472, 2572 within the slot 2461, 2561, further compression of the compression pad 2462, 2562 is permitted, thus decreasing the effective durometer of the compression pad 2462, 2562. Compression pads 2462, 2562 may also define portions of different durometer and/or other properties similar to any of the aspects described above.

With reference to FIGS. 26 and 27, in aspects, rather than slot 161 of jaw member 142 defining relief recesses, the compression pad 2662, 2762 may occupy only a portion of slot 161 in an at-rest position thereof to enable outward (and, in aspects, downward) expansion of the compression pad 2662, 2762 in response to compression thereof from the tissue contacting surface 2668, 2768 of the compression pad 2662, 2762. As the compression pad 2662, 2762 is compressed in this manner, the compression pad 2662, 2762 is urged at least partially into the previously unoccupied portions of slot 161. Thus, the effective durometer of the compression pad 2662, 2762 is decreased. Compression pad 2662, 2762 may also define portions of different durometer and/or other properties similar to any of the aspects described above.

Referring in particular to FIG. 26, compression pad 2662 defines a substantially solid configuration and is disposed within slot 161 to substantially fill the open end of slot 161 while unoccupied pockets of slot 161 are enclosed within slot 161 between the portions of distal body 143b of jaw member 142 defining slot 161 and compression pad 2662, due to the fact that the volume (and shape) of compression pad 2662 is smaller than that of slot 161. Turning in particular to FIG. 27, compression pad 2762 is disposed within slot 161 and defines a hollow interior, e.g., an arch-shaped configuration with a hollow concave interior, such that unoccupied pockets of slot 161 are enclosed within slot 161 between the portions of distal body 143b of jaw member 142 defining slot 161 and compression pad 2762 and within the hollow interior of compression pad 2762.

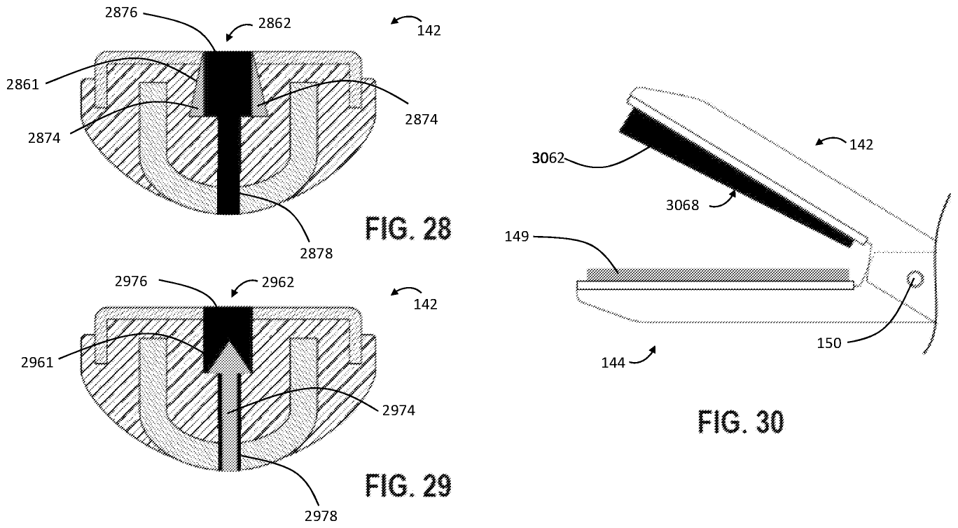

Turning to FIGS. 28 and 29, in aspects, the compression pad 2862, 2962 is formed in multiple stages. For example, the compression pad 2862, 2962 may be formed via a two-shot overmolding or injection molding process, e.g., wherein a first shot forms a first portion 2874, 2974 of the compression pad 2862, 2962 and a second shot forms a second portion 2876, 2976 of the compression pad 2862, 2962. As another example, a first portion 2874, 2974 of the compression pad 2862, 2962 may be pre-formed and the second portion 2876, 2976 of the compression pad 2862, 2962 may then be formed about or within the first portion 2874, 2974 and within the slot 2861, 2961 of jaw member 142, e.g., via overmolding or injection molding. First and second portions 2874, 2974 and 2876, 2976, respectively, may be formed from different materials or otherwise be configured to define portions of different durometer and/or other properties similarly as detailed above.

Referring in particular to FIG. 28, compression pad 2862 may be formed by a first overmolding step forming first portion 2874 including first and second sections lining the lateral walls of slot 2861 on either side thereof and a second overmolding step forming second portion 2876 between the first and second sections of first portion 2874 and filling the remainder of slot 2861. With respect to FIG. 29 in particular, compression pad 2962 may be formed by a first overmolding step forming first portion 2974 including a central body spaced apart from the lateral walls of slot 2961 and recessed relative to the open end of slot 2961 and a second overmolding step forming second portion 2976 surrounding first portion 2974 and filling the remainder of slot 2961. In either of the above aspects, jaw member 142 may include an access aperture 2878, 2978 extending through a bottom surface thereof into communication with the slot 2861, 2961 to enable the injection of the overmold material into slot 161 during at least one of the overmold steps.

Turning to FIG. 30, compression pad 3062 may be configured similar to any of the compression pads detailed hereinabove and configured to extend longitudinally along jaw member 142, e.g., in alignment with cutting electrode 149, and protrude from jaw member 142 towards jaw member 144. Compression pad 3062, more specifically, defines a tissue contacting surface 3068 and tapers in height, e.g., the extent to which tissue contacting surface 3068 protrudes from jaw member 142 towards jaw member 144. For example, as shown, the height of compression pad 3062 may taper in a distal to proximal direction along at least a portion of a length of compression pad 3062 such that tissue contacting surface 3068 is angled proximally along at least a portion of the length of compression pad 3062. This configuration may provide a uniform, or more uniform, grasping pressure applied to tissue grasped between compression pad 3062 and cutting electrode 149 along the lengths of jaw members 142, 144 and/or a uniform, or more uniform, gap distance between jaw members 142, 144 along the lengths of jaw members 142, 144 since the tapering height of compression pad 3062 counteracts the tendency for greater pressures and smaller gap distances towards the pivot 150 as compared to farther from the pivot 150. Other configurations are also contemplated.

While several aspects of this disclosure have been shown in the drawings, it is not intended that this disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A surgical end effector assembly, comprising:
first and second jaw members including respective first and second tissue contacting surfaces, at least one of the first or second jaw members movable relative to the other of the first or second jaw members between a spaced apart position and an approximated position for grasping tissue between the first and second tissue contacting surfaces,
wherein the second jaw member includes a cutting electrode extending from the second jaw member towards the first jaw member, and
wherein the first jaw member includes a compression pad configured to oppose the cutting electrode in the approximated position of the first and second jaw members for grasping tissue between the cutting electrode and the compression pad in the approximated position of the first and second jaw members, the compression pad including a first portion having a first durometer and a second portion having a second durometer different from the first durometer.

2. The surgical end effector assembly according to claim 1, wherein the first and second portions of the compression pad are vertically stacked such that the first portion is configured to contact tissue while the second portion is substantially unexposed within the first jaw member.

3. The surgical end effector assembly according to claim 1, wherein the first and second portions of the compression pad are longitudinally aligned such that the first portion defines a proximal compression pad portion and the second portion defines a distal compression pad portion.

4. The surgical end effector assembly according to claim 1, wherein the first and second portions of the compression pad are laterally aligned such that the first portion defines a right compression pad portion and the second portion defines a left compression pad portion.

5. The surgical end effector assembly according to claim 1, wherein the first portion of the compression pad includes first and second outer sections and wherein the second portion of the compression pad is disposed between the first and second outer sections.

6. The surgical end effector assembly according to claim 1, wherein the first portion includes a body of the compression pad and wherein the second portion includes a plurality of voids defined through the body of the compression pad.

7. The surgical end effector assembly according to claim 6, wherein at least one void of the plurality of voids is filled with a material different from a material forming the body of the compression pad.

8. The surgical end effector assembly according to claim 1, wherein the first portion is a first overmold and the second portion is a second overmold.

9. The surgical end effector assembly according to claim 1, wherein the second portion surrounds at least a portion of the first portion.

10. The surgical end effector assembly according to claim 1, wherein the first portion includes a first filler material and the second portion includes a second, different filler material or no filler material.

11. A surgical end effector assembly, comprising:
first and second jaw members including respective first and second tissue contacting surfaces, at least one of the first or second jaw members movable relative to the other of the first or second jaw members between a spaced apart position and an approximated position for grasping tissue between the first and second tissue contacting surfaces,
wherein the second jaw member includes a cutting electrode extending from the second jaw member towards the first jaw member, and
wherein the first jaw member includes a compression pad disposed within a slot defined through the first tissue contacting surface, the compression pad configured to oppose the cutting electrode in the approximated position of the first and second jaw members for grasping tissue between the cutting electrode and the compression pad with the cutting electrode at least partially compressing the compression pad from an initial condition to a compressed condition in the approximated position of the first and second jaw members,
wherein, in the initial condition, a portion of the compression pad is spaced apart from a wall defining the slot, and
wherein, in the compressed condition, the portion of the compression pad is urged towards and into contact with the wall defining the slot.

12. The surgical end effector assembly according to claim 11, wherein the slot includes at least one relief recess, and wherein the wall defining the slot is disposed within the relief recess.

13. The surgical end effector assembly according to claim 12, wherein the at least one recess includes a pair of relief recesses disposed on either side of the compression pad.

14. The surgical end effector assembly according to claim 10, wherein the wall is a lateral wall of the slot.

15. The surgical end effector assembly according to claim 10, wherein the compression pad defines a volume less than a volume of the slot such that in the initial condition, a pocket is defined between the compression pad and the wall defining the slot.

16. A surgical instrument, comprising:
a housing;
a shaft assembly extending distally from the housing; and
an end effector assembly extending distally from the shaft assembly, the end effector assembly including:
first and second jaw members including respective first and second tissue contacting surfaces, at least one of the first or second jaw members movable relative to the other of the first or second jaw members between a spaced apart position and an approximated position for grasping tissue between the first and second tissue contacting surfaces,
wherein the second jaw member includes a cutting electrode extending from the second jaw member towards the first jaw member,
wherein the first jaw member includes a compression pad configured to oppose the cutting electrode in the approximated position of the first and second jaw members for grasping tissue disposed between the cutting electrode and the compression pad in the approximated position of the first and second jaw members, and
wherein the compression pad defines a varied compressibility in at least one dimension of the compression pad.

17. The surgical instrument according to claim 16, wherein the compression pad includes portions formed from different materials, thereby defining the varied compressibility.

18. The surgical instrument according to claim 16, wherein the compression pad is at least partially disposed within a slot defined within the first jaw member, and wherein the slot defines a varied width to thereby vary a width of the compression pad and define the varied compressibility.

19. The surgical instrument according to claim 16, wherein compression pad is at least partially disposed within a slot defined within the first jaw member, the varied compressibility defined by a first portion of the compression pad extending into a previously unoccupied portion of the slot in response to compression of the compression pad and a second portion of the compression pad being urged against a wall of the slot in response to the compression of the compression pad.

20. The surgical instrument according to claim 16, wherein the varied compressibility at least partially corresponds to a variation in force applied to tissue grasped between the cutting electrode and the compression pad in the approximated position of the first and second jaw members.
